(19) United States
(12) Patent Application Publication
(10) Pub. No.: US 2015/0329825 A1
(43) Pub. Date: Nov. 19, 2015

(54) COMPOSITIONS AND METHODS EMPLOYING STEM CELL-DERIVED CARDIOMYOCYTES

(71) Applicant: THE REGENTS OF THE UNIVERSITY OF MICHIGAN, Ann Arbor, MI (US)

(72) Inventors: Todd Herron, South Lyon, MI (US); José Jalife, Saline, MI (US)

(21) Appl. No.: 14/710,241

(22) Filed: May 12, 2015

Related U.S. Application Data
(60) Provisional application No. 61/992,673, filed on May 13, 2014.

(51) Int. Cl. C12N 5/077

(52) U.S. Cl. CPC C12N 5/0657; C12N 2506/45; C12N 2533/90; C12N 2533/30

(57) ABSTRACT

The present disclosure provides compositions and methods employing stem cell-derived cardiomyocytes. In some embodiments, methods of generating cardiomyocytes from stem cells (e.g., induced pluripotent stem cells (iPS cells or IPSCs) and embryonic stem cells) are provided. In some embodiments, uses of such cells for research, compound screening and analysis, and therapeutics are provided.

The present application claims priority to U.S. Provisional Patent Application Ser. No. 61/992,673, filed May 13, 2014, the disclosure of which is herein incorporated by reference in its entirety.

### STATEMENT REGARDING FEDERALLY SPONSORED RESEARCH OR DEVELOPMENT

The invention was made with government support under grants P01-HL039707 and P01-HL087226 awarded by the National Institutes of Health. The government has certain rights in the invention.

### FIELD

The present disclosure provides compositions and methods employing stem cell-derived cardiomyocytes. In some embodiments, methods of generating cardiomyocytes from stem cells (e.g., induced pluripotent stem cells (iPS cells or IPSCs) and embryonic stem cells) are provided. In some embodiments, uses of such cells for research, compound screening and analysis, and therapeutics are provided.

### BACKGROUND

Stem cells are pluripotent cells with remarkable potential to develop into many different cell types in the body during early life and growth. In addition, in many tissues they serve as a sort of internal repair system, dividing essentially without limit to replenish other cells as long as the person or animal is still alive. There are two types of stem cells: embryonic stem cells and non-embryonic “somatic” or “adult” stem cells. Induced pluripotent stem cells (iPSCs) are adult cells that have been genetically reprogrammed to pluripotent stem cells.

Stem cells carry promises for regenerative medicine and cell therapy, but are also changing the drug discovery and development process. Emergence of stem cell technologies provides new opportunities to build innovative cellular models. Stem cell models offer new opportunities to improve the manner in which pharmaceutical companies identify lead candidates and bring new drugs to the market. In spite of promising applications, new competencies surrounding stem cell differentiation and proliferation, induction of pluripotent stem cells and creation of efficacy assays are needed to make successful use of stem cells in drug discovery.

Beyond improved models, pluripotent stem cells technologies are introducing applications that were previously not possible. Currently, human clinical populations are poorly represented in drug development with a lack of genetic heterogeneity in human cellular models and a limited number of human disease models. As a result of induced pluripotent stem cell (iPSC) technology, new cellular models can be created from individuals with a diverse range of drug susceptibilities and resistances, offering the promise of a “clinical trial in a dish” in a field where a personalized medicine approach is becoming increasingly predominant.

Despite these advantages there are still several challenges in using stem cells in drug discovery. The differentiation and reprogramming strategies are not standardized and are often based on growth factors, making protocols expensive, poorly reproducible and limited in terms of scale-up. The pace of stem cell research—for example, a single differentiation or reprogramming experiment currently can take more than a month—is too slow to fit into timelines required by the industry. In addition, before pharmaceutical companies typically will invest in the development of such platforms, further demonstrations of success and potential applications are necessary. And last but not least, stem cell culture and differentiation need to be adapted to the high-throughput environment of drug discovery by developing standardized high-throughput and miniaturized assays for in vitro screening.

### SUMMARY

The present disclosure provides compositions and methods employing stem cell-derived cardiomyocytes. In some embodiments, methods of generating cardiomyocytes from stem cells (e.g., induced pluripotent stem cells (iPS cells or IPSCs) and embryonic stem cells) are provided. In some embodiments, uses of such cells for research, compound screening and analysis, and therapeutics are provided.

The generation of human induced pluripotent stem cell-derived cardiomyocytes (hiPSC-CMs), as provided herein, offers a revolutionary platform to transform the way heart disease is treated and to provide novel mechanistic insight into physiological and pathophysiological processes. Existing methods lead to generation of hiPSC-CMs with slow conduction velocities (CVs) that can be attributed to the immature structural and functional phenotype of the hiPSC-CMs generated to date. Experiments conducted with the compositions and methods described herein demonstrates that induced pluripotent stem cell-derived cardiomyocytes (hiPSC-CM) had a mature electrophysiological phenotype with more negative resting membrane potentials, faster action potential upstrokes and faster CVs. Thus, in some embodiments, provided herein are compositions and methods to obtain mature stem cell derived cardiovascular cells for research (e.g., patient-specific disease modeling), drug (and other therapy) discovery, and therapeutic applications (e.g., autologous cellular therapies).

Among other advantages, the cells provide herein offer (e.g., as compared to prior available stem-cell derived cardiomyocytes): a) mature cellular structure/function; b) faster maturation rate; c) amenability to high throughput screening platforms; d) a viable personalized drug testing platform; and e) a disease-specific drug testing platform. Uses include, but are not limited to, drug discovery, cardio-toxicity testing, high throughput toxicity testing, disease-specific drug testing, personalized medicine, regenerative medicine, and research models.

Animal models are currently the best relevant physiological systems to evaluate drug efficacy and toxicity, but cost, time and ethical issues associated with in vivo studies—as well as the uncertainty of translatability to humans—emphasize the advantages of relevant in vitro models. The U.S. Food and Drug Administration (FDA) is placing added importance on in vitro testing, recommending the use of human cell lines to characterize drug metabolic pathways. Although immortalized cell lines have their advantages, they are not the best representation of normal tissue, so right now primary cells are the gold standard for in vitro toxicity testing. Yet, human primary cells present significant challenges, including cost, limited or no accessibility for particular cell types—such as cardiomyocytes—short life span and loss of function in vitro, which prevents chronic toxicity studies.

The present disclosure provides biologically relevant cardiomyocytes, which permit in vitro analysis and development of compounds for diagnosis and treatment of cardiovascular diseases and provides cells useful for treatment of cardiovascular diseases. In 2011, an estimated 880 million cases of cardiovascular diseases were reported globally. The U.S. and Europe face high and increasing rates of cardiovascular disease, with heart disease now representing roughly 30% of all deaths. The number of procedures performed on people with cardiovascular diseases using stem cell therapies is estimated to reach over 4 million in a best case scenario by 2020 and revenues from proprietary stem cell therapies for cardiovascular diseases is expected to reach $8 billion in 2020. Cardiovascular diseases comprise 14.9% of the total stem cell therapy market.

In some embodiments, provided herein are methods for preparing (e.g., functionally mature or electrophysiologically mature) cardiomyocytes (e.g., from stem cells), comprising: culturing cells (e.g., stem cells, pluripotent cells, non-terminally differentiated cells (regardless of pluripotency), induced pluripotent stem cell derived cardiomyocytes) on a flexible surface coated with extracellular matrix proteins under conditions such that the cells generate cardiomyocytes (e.g., mature cardiomyocytes). In some embodiments, such culturing comprises cellular differentiation of cells into cardiomyocytes. In some embodiments, growth factors are provided with the extracellular matrix proteins. In some embodiments the culturing comprises culturing an interconnected monolayer of the cells (e.g., comprising greater than 50,000, 75,000, 100,000, or 125,000 cells per monolayer). In some embodiments, the stem cells are induced pluripotent stem cells (iPSCs) (e.g., human iPSCs). In some embodiments, the stem cells are embryonic stem cells (e.g., embryonic human stem cells).

A wide variety of flexible (e.g., pliable) surfaces may be employed. In some embodiments, the flexible surface is a coverslip. The flexible surface may be made of any of a variety of different materials including, but not limited to, plastics, rubbers, ceramics, membranes, synthetic or natural tissues, etc. In some embodiments, the flexible surface is a silicone film (e.g., polydimethylsiloxane (PDMS) film). In some embodiments, such surfaces are provided in devices having multiple segregated regions (e.g., multi-well plates, e.g., 6-well, 12-well, 24-well, 48-well, 96-well, 384-well, 1536-well, etc.).

In some embodiments, the extracellular matrix proteins are provided by a MATRIGEL coating (a gelatinous protein mixture screted by Engelbreth-Holm-Swarm (EHS) mouse sarcoma cells marketed by Corning Life Sciences and by Trevigen, Inc. under the name Cultrex BME; this mixture resembles the complex extracellular environment found in many tissues).

In some embodiments, the culturing and differentiation of stem cells occurs in a period less than 2 weeks (e.g., less than 1 week, within a period of 4 days) of culturing.

In some embodiments, the cardiomyocytes produced have one or more properties resembling natural (in vivo) cardiomyocytes and/or have superior characteristics as compared to the same cells cultured in a different context (e.g., without a flexible surface, without a surface coated with extracellular matrix proteins). Such characteristics can be quantitatively or qualitatively assessed using assays described herein and comparisons made to control samples (e.g., natural cardiomyocytes or stem cells differentiated using an alternative method such as any of the prior inferior methods cited herein). For example, in some embodiments, the generated cardiomyocytes have one or more (in any combination) or all of the following properties: a high action potential upstroke velocity (e.g., greater than or equal to 75 V/s, 100Vs, 150 V/s); a hyperpolarized diastolic membrane potential; a high propagation velocity (e.g., greater than 25 cm s⁻¹, greater than 30 cm s⁻¹, greater than 40 cm s⁻¹, greater than 50 cm s⁻¹); elevated current density (relative to control) from single cell patch clamp analysis of I_{Na}; elevated inward rectifier potassium current density (I_{K1}) (relative to control); and elevated expression of cardiomyocyte marker proteins (e.g., Kir2.1, SCN5A, Cx43, and Kindlin-2).

In some embodiments, the cells are genetically altered (e.g., prior to or following culturing and/or differentiation). For example, a transgene may be added to provide a detectable marker (for research and drug screening application), to assist with transplantation (e.g., increase integration or decrease rejection), or to provide a therapeutic benefit (e.g., growth factor expression, etc.).

Further provided herein are cardiomyocytes produced by any of the methods. In some embodiments, the produced cardiomyocytes have one or more or all of the above recited properties or characteristics.

Also provided herein are methods, devices, and systems for using such cells for research, drug screening, diagnostic, and therapeutic applications. For example, in some embodiments, provided herein are high-throughput screening methods, devices and system that permit multiple of such cell compositions to be tested in parallel (e.g., in a device comprising a plurality of chambers (e.g., wells) containing such cells). In some embodiments, a membrane comprising such cells is provided, for example, for transplantation to a subject for research, diagnostic, or therapeutic purposes. In some embodiments, screening methods comprise: a) providing a cell composition described above; b) exposing a test compound (e.g., a candidate therapeutic compound) to the composition; and c) determining an effect of the test compound on said composition. In some embodiments, the effect is one or more cardiac electrophysiological functions including, but not limited to, action potential duration, beating frequency, conduction velocity or intracellular calcium flux amplitudes.

In some embodiments, kits are provided. In some embodiments the kits comprise components necessary, useful, or sufficient for practicing methods described herein (e.g., comprising one or more or all of a flexible surface, a coating for the flexible surface, stem cells, differentiation factor, devices or reagents for analyzing the cells for any of the above recited characteristics, and positive and negative controls). In some embodiments, kits comprise components associated with the use of the cells (e.g., cardiomyocytes, drug screening devices and reagents, devices and reagents for characterizing cardiomyocytes, components for transplantation or other administration of cardiomyocytes therapeutically).

In some embodiments, the present disclosure provides a kit or system comprising: cultured induced pluripotent stem cell-derived cardiomyocytes on a flexible culture substrate coated with extracellular matrix proteins. In some embodiments, the cardiomyocytes are cultured from iPSCs or stem cells.

In some embodiments, compositions comprising cultured induced pluripotent stem cell-derived cardiomyocytes, wherein said cardiomyocytes have at least one property selected from, for example, a mature electrophysiological phenotype with more resting membrane potentials, faster action potential upstrokes, and faster CVs (e.g., relative to prior available stem cell-derived cardiomyocytes) are provided.

### DESCRIPTION OF THE FIGURES

FIG. 1 shows electrical wave propagation in mature hiPSC-CM monolayers. A. Left, optical activation map of spontaneously initiated electrical wave propagation in an hiPSC-CM cardiomyocyte monolayer cultured on PDMS+matrigel. Right, single pixel signals of optical action potentials recorded from the pacemaker site and a more distal site in the monolayer. B, Action potential impulse propagation velocity slowed as pacing frequency increased. C, Action potential duration calculated at 80% repolarization (APD₈₀) shortened as pacing cycle length shortened.

FIG. 2 shows effects of ECM on hiPSC-CM Monolayer Impulse Propagation. A, Four different ECM combinations were tested to determine the effects on hiPSC-CM monolayer structure and function. B, Activation maps of calcium impulse propagation in the different plating conditions. C, Quantification of impulse propagation. D, Quantification of impulse propagation during electrical stimulation at 1 Hz.

FIG. 3 shows mature hiPSC-CM action potential and sodium channel characteristics. A, Representative action potential recordings from monolayers plated on fibronectin on glass (left) and matrigel on PDMS (right). B-E, AP parameters demonstrate significant electrophysiological maturation of monolayers plated on matrigel on PDMS. F, Representative I_{Na} recordings of hiPSC-CMs cultured on fibronectin on glass and cardiomyocytes cultured on matrigel on PDMS. G, Current-Voltage (I-V) relationship of sodium current in each condition shows elevated I_{Na} in cardiomyocytes culture on matrigel coated PDMS.

FIG. 4. shows potassium current density (I_{K1}) in hiPSC-CM single cells. A, Representative I_{K1} recordings in single hiPSC-CM cultured on fibronectin on glass and hiPSC-CM cultured on matrigel on PDMS. B, I-V relationship of I_{K1} in each condition shows significantly elevated current density in hiPSC-CM cultured on matrigel on PDMS. C, Western blot probing for Kir2.1 demonstrates expression only in hiPSC-CMs cultured on matrigel on PDMS (lane 1 in each blot, 2 individual monolayers for each condition).

FIG. 5 shows hiPSC-CM response to I_{Kr} blockade using E4031 (100 nmol L⁻¹). A, intracellular calcium flux measurements in hiPSC CMs cultured on rigid plastic bottom dishes (matrigel coated) show very significant impact of E4031 blockade on spontaneous beating frequency and calcium transient duration 80 (CaTD₈₀). B, representative traces from one PDMS bottom well shows more modest effect of E4031 on beat frequency and CaTD₈₀ in this condition, which indicates the presence of other repolarizing currents to compensate for the blockade of I_{Kr}. C, quantification shows greater effect of E4031 on the beat frequency and CaTD₈₀ in immature iPSC CMs cultured on rigid plastic bottom dishes compared to PDMS bottom dishes.

FIG. 6 shows ECM Effect on Cx43 Expression. A, Immunostaining of α-actinin and Cx43. B, Western blotting for Cx43 and total myosin confirms the immunofluorescence results of panel A. C, Cx43 expression is also promoted in hESC-CM monolayers cultured on PDMS as opposed to the same cells plated on glass coverslips.

FIG. 7 shows maturation of BJ-hiPSC-CM monolayers. A, The cardiomyocyte specific SIRPA2a antigen was used to purify BJ-hiPSC-CMs by magnetic activated cell sorting (MACS) following cardiac differentiation. B, (top panel) immunostaining for Ki67 and α-actinin shows decreased proliferative activity in BJ-hiPSC-CMs cultured on PDMS compared to glass coverslips (0.87±0.29 CM/60×field compared to 6.2±0.90 CM/60×field; n=8 and n=10. *P=0.0001). B, (Bottom panel), immunostaining for sarcomeric actin and N-cadherin shows hypertrophy and elongation of BJ-iPSC-CMs cultured on PDMS compared to glass coverslips (cell area=3,678.59±171.6 μm² compared to 2,071.44±116.7 μm²;n=84 and n=83. *P=0.000003). C, Myofilament maturation is shown by Western Blotting for cTnI protein expression.

FIG. 8 shows integrin signalling via Focal Adhesion Kinase in mature hiPSC-CM monolayers. A, RT-PCR analysis indicates elevated expression of ITGA5 and ITGB1 integrin receptor genes in mature monolayers (red). RT-PCR performed in triplicate for 5 individual monolayers for each group. B, PTK2 gene expression is elevated in mature monolayers. C, Pharmacological inhibition of FAK activity using FAK inhibitor-14 prevents expression of mature myofilament protein isoforms including cTnI and β-MyHC. D, FAK inhibition prevents PDMS induced hypertrophic growth of hiPSC-CMs.

FIG. 9 shows maturation of hiPSC-CMs differentiated on PDMS. A, Activation maps of calcium impulse propagation using 19-9-11T hiPSC-CM monolayers on day 30 of differentiation show faster conduction when iPSCs are differentiated on PDMS coverslips rather than on plastic bottom dishes. B, Cardiac monolayers differentiated on PDMS had significantly faster conduction velocity compared to those differentiated on plastic bottom dishes (48.2 2.68 cm s−1 compared to 25.4 1.64 cm s-1, n=11 and n=7 monolayers; *P=0.00001).

FIG. 10 shows I_{Na} activation and inactivation profiles for each condition.

FIG. 11 shows 96 well optical mapping technique.

FIG. 12 shows hiPSC CM response to E4031 depends on the hardness of the underlying substrate (plastic vs. PDMS). A, The average reduction of spontaneous beating frequency after I_{Kr} blockade was less in hiPSC CMs cultured on matrigel coated PDMS compared to plastic bottom dishes. B, Similarly the average E4031 induced increase of CaTD₈₀ was greater in monolayers cultured on plastic bottom dishes compared to PDMS bottom dishes.

FIG. 13 shows that hiPSC-CM monolayer cell size was determined 5 days post thaw by immunoflurescent staining for N-cadherin (A). B, Specifically, there was no difference in hiPSC-CM size between the fibronectin+glass group and the matrigel+glass group (938.7±61.2 μm², n=79 vs. 917.8±64.2 μm², n=66). C, Western blotting for kindlin-2 in each ECM condition. D, Quantification of kindlin-2 protein levels in monolayers cultured on fibronectin on glass compared to matrigel on PDMS (n=4 monolayers per group). ANOVA Bonferroni's multiple comparisons test, **P=0.0009

FIG. 14 shows hiPSC-CM monolayers were cultured on Glass or PDMS coverslips with or without the indicated concentrations of FAK inhibitor-14.

FIG. 15 shows that focal adhesion kinase (FAK) activity is required for maturation process.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

As used herein, the term “treatment” is defined as the application or administration of a therapeutic agent described herein (e.g., composition comprising cardiomyocytes) to a patient, or application or administration of the therapeutic agent to an isolated tissue or cell line from a patient, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

As used herein, the term “functionally mature cardiomyocytes” refers to cardiomyoctes (e.g., prepared using the methods described herein) that exhibit one or more properties of primary cardiomyocytes (e.g., electrophysiological properties described herein). In some embodiments, “functionally mature cardiomyocytes” are also referred to as “electrophysiologically mature cardiomyocytes.”

The term “administration” and variants thereof (e.g., “administering” a compound) in reference to cells or a compound mean providing the cells or compound or a prodrug of the compound to the individual in need of treatment or prophylaxis. When cells or a compound of the technology or a prodrug thereof is provided in combination with one or more other active agents, “administration” and its variants are each understood to include provision of the compound or prodrug and other agents at the same time or at different times. When the agents of a combination are administered at the same time, they can be administered together in a single composition or they can be administered separately. As used herein, the term “composition” is intended to encompass a product comprising the specified ingredients in the specified amounts, as well as any product that results, directly or indirectly, from combining the specified ingredients in the specified amounts.

By “pharmaceutically acceptable” is meant that the ingredients of the pharmaceutical composition are compatible with each other and not deleterious to the recipient thereof.

The term “subject” as used herein refers to an animal, preferably a mammal, most preferably a human, who has been the object of treatment, observation, or experiment.

The term “effective amount” as used herein means that amount of an agent (e.g., cardiomyocytes) that elicits the biological or medicinal response in a cell, tissue, organ, system, animal, or human that is being sought by a researcher, veterinarian, medical doctor, or other clinician. In some embodiments, the effective amount is a “therapeutically effective amount” for the alleviation of the symptoms of the disease or condition being treated. In some embodiments, the effective amount is a “prophylactically effective amount” for prophylaxis of the symptoms of the disease or condition being prevented.

“Feeder cells” or “feeders” are terms used to describe cells of one type that are co-cultured with cells of another type, to provide an environment in which the cells of the second type can grow. When a cell line spontaneously differentiates in the same culture into multiple cell types, the different cell types are not considered to act as feeder cells for each other within the meaning of this definition, even though they may interact in a supportive fashion. “Without feeder cells” refers to processes whereby cells are cultured without the use of feeder cells.

A cell is said to be “genetically altered” when a polynucleotide has been transferred into the cell by any suitable means of artificial manipulation, or where the cell is a progeny of the originally altered cell that has inherited the polynucleotide. The polynucleotide will often comprise a sequence encoding a protein of interest, which enables the cell to express the protein at an elevated level. The genetic alteration is said to be “inheritable” if progeny of the altered cell have the same alteration.

### DETAILED DESCRIPTION

The present disclosure provides compositions and methods employing stem cell-derived cardiomyocytes. In some embodiments, methods of generating cardiomyocytes from stem cells (e.g., induced pluripotent stem cells (iPS cells or IPSCs) and embryonic stem cells) are provided. In some embodiments, uses of such cells for research, compound screening and analysis, and therapeutics are provided.

Experiments conducting during the development of embodiments of the technology discovered that culturing of cells on flexible (e.g., pliable) supports comprising extracellular matrix (ECM) generates differentiated cardiomyocytes that more closely resemble natural cardiomyocytes than prior techniques permitted. For example, the following was demonstrated in hiPSC-CM monolayers generated by the differentiation methods described herein: First, culturing the monolayers on a flexible membrane coated, for example, with matrigel, alone increases the impulse CV to values as high as 55 cm s⁻¹, which is 2× faster than previously reported for human iPSC-CM monolayers (Lee P, et al., Circulation Research. 2012;110:1556-1563, herein incorporated by reference in its entirety); Second, the flexible membrane/ECM promotes electrophysiological maturation of the hiPSC-CM single cell increased inward rectifier potassium and sodium inward current densities, giving rise to a well polarized MDP and action potential upstroke velocity, respectively. Third, formation of intercellular gap junctions and mechanical junctions are promoted by the flexible membrane/ECM. Fourth, hiPSC-CM hypertrophy is induced when plated on flexible membrane (e.g., pliable PDMS) rather than on rigid glass coverslips. Remarkably, the maturation of hiPSC-CMs plated on the optimal biomatrix combination as reported here occurs in just four days. This represents a major advance over previous reports that have demonstrated modest maturation of stem cell derived cardiomyocytes over a period of up to two to four months (Zuppinger C, et al., Journal of Molecular and Cellular Cardiology. 2000;32:539-555; and Li F, et al., J Mol Cell Cardiol. 1996;28:1737-1746, herein incorporated by reference in their entireties).

Combining ECM (e.g. matrigel) with softer synthetic biomaterials (e.g., flexible materials) provides an avenue to produce mature hiPSC and/or embryonic stem cell (ESC) derived human myocytes and electrically coupled monolayers that are more useful for disease modeling, drug testing and for cardiac regeneration applications. The vast majority of studies to date using hiPSC-CMs to model inherited cardiomyopathies have performed phenotype analysis exclusively on single isolated cardiomyocytes, which poses a limitation in that cardiomyocytes do not normally exist in nature on their own, but rather exist as a functional syncytium of interconnected cells that beat as a functional unit. For example, studies on patient specific iPSC models of inherited cardiac diseases such as hypertrophic cardiomyopathy (HCM) (Katzberg A A, et al., American Journal of Anatomy. 1977;149:489-499, herein incorporated by reference in its entirety), catecholaminergic polymorphic ventricular tachycardia (CPVT) (Spach M S, et al., Circulation Research. 2000;86:302-311, herein incorporated by reference in its entirety), and arrhythmogenic right ventricular cardiomyopathy (ARVC) (Montanez E, et al., Genes & Development. 2008;22:1325-1330, herein incorporated by reference in its entirety) have not examined electrical impulse propagation using electrically coupled monolayers of cardiomyocytes. These diseases would be better modeled and arrhythmia mechanisms would be better defined using electrically coupled monolayers to complement single cell analysis. This is especially true for inherited disease such as ARVC caused by gene mutations of desmosomal proteins present at the cell-cell junctions such as plakophillin2 and plakoglobin. For cardiac arrhythmia research, the development of mature hiPSC-CM monolayers also offers many advantages over rodent model systems that are commonly used in many laboratories. The mature electrophysiological phenotype with elevated inward rectifier potassium and rapid sodium inward currents that yield more negative MDPs, more rapid action potential upstrokes, and faster impulse conduction velocities in hiPSC-CM monolayers plated on flexible surface+ECM represent a significant advance toward the use of these cells for patient-specific disease modeling, drug testing and for autologous cellular therapies. For example, cell may be used with an implantable cardiac patch to improve pump function to ischemic hearts. Ideally an implantable autologous cardiac patch integrates into the native heart tissue and replaces necrotic scar tissue to rescue failing hearts. A concern with this approach, however, is the introduction of a pro-arrhythmic substrate into the heart caused by slow impulse propagation of previously developed cardiac patches. Generation of cardiac patches with rapid impulse propagation as provided herein is contemplated as useful to augment ischemic hearts without introducing a pro-arrhythmic substrate (i.e., slow conducting patch).

### Cells

A wide variety of cells and stem cells may be employed with the technology described herein. In some embodiments, the cell is a pluripotent cell with potential for cardiomyocyte differentiation. Such cells include embryonic stem cells and induced pluripotent stem cells, regardless of source. For example, induced pluripotent stem cells may be derived from stem cells or adult somatic cells that have undergone a dedifferentiation process.

Induced pluripotent stem cells may be generated using any known approach. In some embodiments, iPSCs are obtained from adult human cells (e.g., fibroblasts). In some embodiments, modification of transcription factors (e.g., Oct3/4, Sox family members (Sox2, Sox1, Sox3, Sox15, Sox18), K1f Family members (K1f4, K1f2, K1f1, K1f5), Myc family members (c-myc, n-myc, 1-myc), Nanog, LIN28, Glis1, etc.) or mimicking their activities is employed to generate iPSCs (using transgenic vector (adenovirus, lentivirus, plasmids, transposons, etc.), inhibitors, delivery of proteins, microRNAs, etc.).

In some embodiments the cells are non-terminally differentiated cells (regardless of pluripotency) or other non-maturated cells.

In some embodiments, cells are screened for propensity to develop teratomas or other tumors (e.g., by identifying genetic lesions associated with a neoplastic potential). Such cells, if identified, are discarded.

### Culturing/Differentiation

Stem cells are cultured and/or differentiated on a flexible (e.g., soft, pliable) surface coated with ECM proteins. In some embodiments, the culture platform is selected based on its ability to produce cells of equivalent quality as those generated with the matrigel on PDMS of the experimental examples described below. As such, the flexibility of the surface is such that cells having one or more of the desired properties herein are generated. Likewise the constituents of the ECM are selected to achieve the same result.

Culture conditions are selected based on the cells employed. In some embodiments, the conditions used are those of Lee et al., 2012, supra. In some embodiments, the process comprises thawing (if cryopreserved) and plating iPSCs on the coated support at a desired density (e.g., 125,000 cells per monolayer) in differentiation media (e.g., embryoid body differentiation media, commonly referred to as embryoid body-20, comprising 80% Dulbecco Modified Eagle Medium (DMEM/F12), 0.1 mmol/L⁻¹ nonessential amino acids, 1 mmol/L⁻¹ L-glutamine, 0.1 mmol/L⁻¹ β-mercaptoethanol, and 20% fetal bovine serum; Gibco) supplemented with 10 μmol/L⁻¹ blebbistatin. After 24 hours in embryoid body-20, the medium is switched to iCell maintenance medium (Cellular Dynamics), supplemented with 10 μmol/L⁻¹ blebbistatin, and cells are cultured for an additional time period (e.g., 96 hours) at 37° C., in 5% CO₂, with the medium changed once daily.

### Uses

Cardiomyocytes provided herein find use in a variety of research, diagnostic, and therapeutic applications.

In some embodiments, the cells are used for disease modeling and drug development. The quality of the cells and the ability to generate them in a short period of time makes them ideally suited for such research uses, particularly high-throughput analysis. Agents (e.g., antiarrhythmic agents) are contacted with the cells to determine the effect of the agent. Cell may also be modified to include a marker and used either in vitro or in vivo as diagnostic compositions to assess properties of the cells in response to changes in the in vitro or in vivo environment.

Cells also find use in therapeutic approaches, including, but not limited to, transplantation of the cells into a subject (e.g., for tissue repair, to prevent or treat a disease or condition) or organ synthesis.

In some embodiments, cells are used in drug testing applications. For example, in some embodiments, drugs or biological agents are tested. Indications for drug testing include any compound or biological agent in the pharmaceutical discovery and development stages, or drugs approved by drug regulatory agencies, like the US Federal Drug Agency. All classes of drugs, ethical, over-the-counter and nutraceuticals for any medical indications, such as but not limited to, drugs for treating cancer, neurological disorders, fertility, vaccines, blood pressure, blood clotting , immunological disorders, anti-infectives, anti-fungals, anti-allergens, and cardiovascular related disorders.

In some embodiments, drug testing applications determine the effects of new chemical entities on cardiac electrophysiological function including, but not limited to, action potential duration, beating frequency, conduction velocity and intracellular calcium flux amplitudes. Such assays serve to inform drug development businesses on the risk of a compound to cause fatal cardiac arrhythmia or other heart-related side effects. These tests may be acute or performed following long term exposure to a drug.

Embodiments of the present disclosure provide kits comprising the cells described herein. For example, in some embodiments, kits comprise cells (e.g., cardiomyocytes or iPSC or stem cells suitable for differentiating into cardiomyocytes) in or on a flexible surface (e.g., multi-well plate or other surface). In some embodiments, kits further comprise reagents for differentiation or use of cells (e.g., buffers, test compounds, controls, etc.).

### EXAMPLES

Unless specified otherwise, the following experimental techniques were used in the Examples.

Human iPSC-CM Monolayers

Cryopreserved vials (liquid nitrogen) of iCell™ human cardiac myocytes were obtained from Cellular Dynamics International, Inc. (Madison, Wisc.). iCell™ cardiac myocytes are highly purified (>98%) hiPSC derived cells that are cryopreserved after 30-31 days of cardiac directed differentiation. Cells were thawed and subsequently plated on bovine fibronectin-coated (20 μg/mL; Life Technologies) glass coverslips, on bovine fibronectin-coated (20 μg/mL; Life Technologies) transparent PDMS membranes, matrigel (500 μg/mL; BD biosciences) coated glass coverslips, or matrigel (500 μg/mL; BD biosciences) coated transparent PDMS membranes at a density of 125,000 cells per monolayer in differentiation media (see e.g., Zhang J, et al., Circulation Research. 2009;104:e30-e41; and Lee P, et al., Circulation Research. 2012;110:1556-1563, herein incorporated by reference in their entireties). PDMS silicone sheeting was obtained from SMI (Saginaw, Mich.) with 40D (D, Durometer) hardness and cut to 18×18 mm coverslips. After 24 hours, the media was switched to RPMI (Life Technologies) supplemented with B27 (Life Technologies). The cells were cultured for 72 additional hours at 37° C., in 5% CO₂ before phenotype analysis. Thus, hiPSC-CM used were 33-34 days old (from the start of differentiation) at the time of phenotype analysis. In parallel experiments the effect of extracellular matrix on NRVM (Neonatal Rat Ventricular Myocyte) monolayer CV was tested. Isolation and culture techniques for NRVM and monolayer formation was done as described before and described (see e.g., Lee, 2012, supra; Rohr S, et al., Circulation Research. 1991;68:114-130; Munoz V, et al., Circ Res. 2007;101:475-483; and Fast V, et al., Circulation Research. 1994;75:591-595, herein incorporated by reference in their entireties).

### Optical Mapping of Calcium Waves and Action Potentials

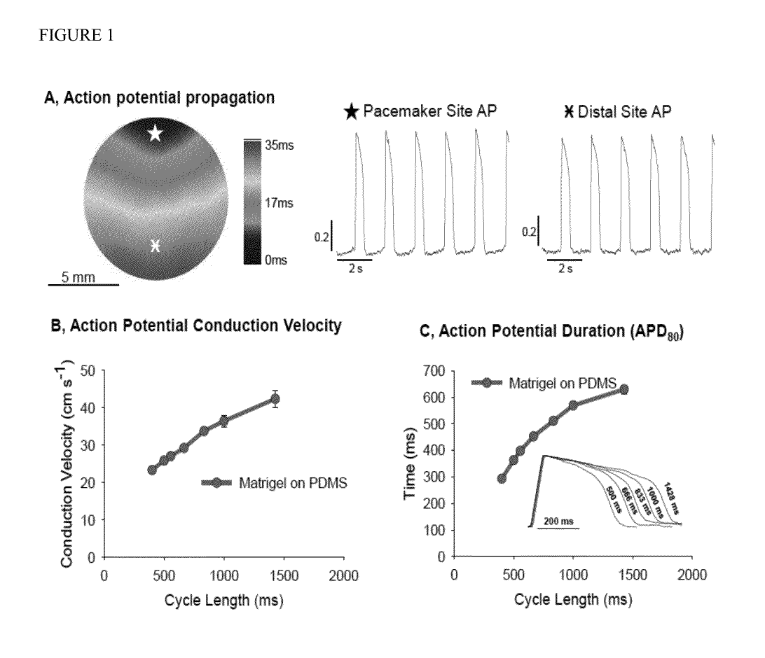

Briefly, monolayers were loaded with the intracellular Ca²⁺ indicator, rhod-2AM (10 μmol L-1, Life Technologies). After a 30 minute incubation time, the cells were washed in Hanks balanced salt solution (HBSS, Life Technologies) for an additional 30 minutes before optical mapping recordings. FIG. 1 shows optical mapping of action potentials using a membrane voltage indicator (FluoVolt, Life Technologies). All human cardiac monolayers displayed pacemaker activity and the spontaneous calcium waves were recorded using a CCD camera (Red-Shirt Little Joe, Scimeasure, Decatur, Ga., 200 fps, 80×80 pixels) with the appropriate emission filter and LED illumination for rhod-2 or FluoVolt fluorescence (Lee P, et al., Heart Rhythm. 2011;8:1482-1491, herein incorporated by reference in its entirety). Movies were filtered in both the time and space domain and conduction velocity (CV) was measured as described previously (Hou L, et al., Circulation Research. 2010;107:1503-1511, herein incorporated by reference in its entirety).

### Action Potential Recordings Using Microelectrodes

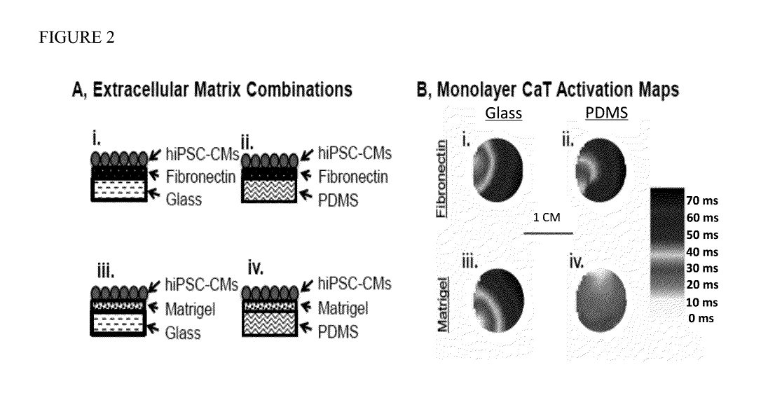

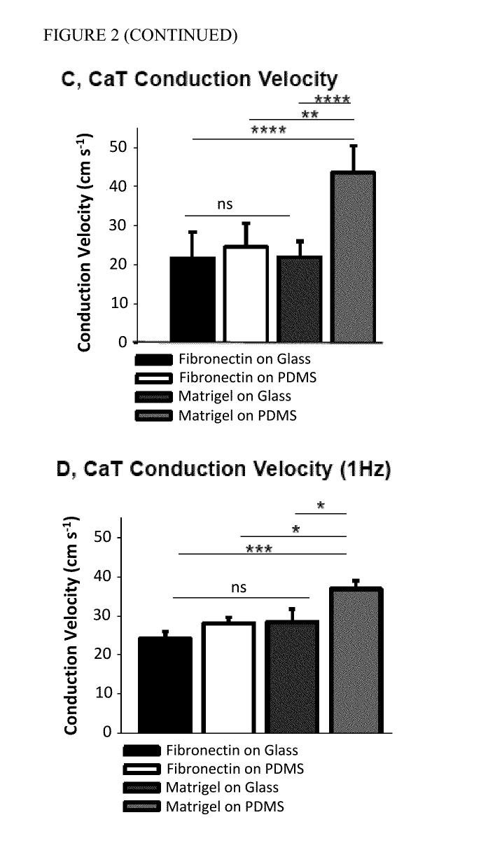

Next, the electrophysiological phenotype was quantified only in two groups of the ECM combinations in order to focus on two extremes of ECM and surface rigidity: fibronectin on glass hiPSC-CM phenotype was compared to matrigel on PDMS phenotype of hiPSC-CMs (FIG. 1: condition i vs. condition iv). Action potentials were recorded from hiPSC-CM monolayers using the current-clamp mode of the MultiClamp 700B amplifier and the Digidata 1440A digitizer (Molecular Devices, Sunnyvale, Calif.). Borosilicate glass pipettes of resistances ranging from 4 to 6 MΩ were filled with an intracellular pipette solution containing (in mmol/L): MgCl₂ (1), EGTA (1), KCl (150), HEPES (5), phosphocreatine (5), K2ATP (4.46), β-hydroxybutyric acid (2), and adjusted to a pH of 7.2 with KOH. Monolayers were perfused with a warm (37±1.5° C.) external solution adjusted to pH 7.4 with NaOH, which contained (in mmol/L): NaCl (148), NaH2PO4 (0.4), MgCl2 (1), glucose (5.5), KCl (5.4), CaCl2 (1.8) and HEPES (15) (Milstein M L, et al., Proceedings of the National Academy of Sciences. 2012;109:E2134-E2143, herein incorporated by reference in its entirety). After the formation of a GΩ seal, the patch membrane was ruptured and spontaneous action potentials were recorded. Data acquisition was performed using pCLAMP software (version 10.3; Molecular Devices, Sunnyvale, Calif.). Action potential recordings were made in different regions of the monolayer (3-5 per monolayer). Using a custom made software, action potential properties including, maximum diastolic potential (MDP), dV/dtmax, overshoot, action potential (AP) amplitude, take-off potential, action potential duration (APD) at 30, 50, 70, 80 and 90% of repolarization were analyzed by calculating the mean values from 10 stable APs in each region. The results were corrected for the calculated junction potential (−8 mV). Representative action potential recordings are in FIG. 2.

Single hiPSC-CM Sodium and Potassium Current Recordings (I_{Na} & I_{K1})

I_{Na} and I_{K1} properties were studied using standard patch clamp techniques (Hamil O P M A, et al., Pflugers Arch. 1981;391:85-100, herein incorporated by reference in its entirety). Single iPSC-CMs were dissociated from monolayers using gentle mechanical agitation in iPSC-CM maintenance medium. Experiments were carrying out using a Multiclamp 700B or Axopatch 200B amplifier (Molecular Devices). Data were acquired and analyzed using the pCLAMP 10.0 Suite of programs (Axon Instruments). Borosilicate glass electrodes were pulled with a Brown-Flaming puller (model P-97), yielding appropriate tip resistances when filled with pipette solution to enable proper voltage control.

Sodium current (INa) in hiPSC-CM were recorded at room temperature (21-22° C.) with pipette resistances <3 MΩ when filled with pipette filling solution containing 5 mmol/L NaCl, 135 mmol/L CsF, 10 mmol/L EGTA, 5 mmol/L MgATP, and 5 mmol/L Hepes, pH 7.2. Extracellular solution contained 20 mmol/L NaCl, 1 mmol/L MgCl₂, 1.8 mmol/L CaCl2, 0.1 mmol/L CdCl₂, 11 mmol/L glucose, 132.5 mmol/L CsCl, and 20 mmol/L Hepes, pH 7.35. Appropriate whole-cell capacitance and series resistance compensation (≧70%) was applied along with leak subtraction. To assess the I_{Na} density, cells were held at −120 mV and stepped to various test potentials (−100-55 mV in 5-mV increments, 200-ms duration, 2,800-ms interpulse intervals). Voltage-dependent activation of I_{Na} was assessed by generating conductance voltage relationships (m-infinity curves) and fitting the data with a standard Boltzman function (Origin 8.1; Microcal). Voltage-dependence of inactivation was assessed by holding at −140 to −40 mV followed by a 30-ms test pulse to −40 mV to elicit I_{Na} (Milstein M L, et al., Proceedings of the National Academy of Sciences. 2012;109:E2134-E2143; and Sato P Y, et al., Circulation Research. 2009;105:523-526, herein incorporated by reference in their entireties). Inward rectified potassium current (I_{K1}) in hiPSC-CM was recorded at room temperature (21-22° C.) with pipette resistances 3-4 MΩ filled with the following standard pipette filling solution: 148 mmol/L KCl, 1 mmol/L MgCl₂, 5 mmol/L EGTA, 5 mmol/L Hepes, 2 mmol/L creatine, 5 mmol/L ATP, 5 mmol/L phosphocreatine, pH 7.2, with KOH. The standard external solutions contained 148 mmol/L NaCl, 0.4 mmol/L NaH₂PO₄, 1 mmol/L MgCl₂, 5.4 mmol/L KCl, 1.8 mmol/L CaCl₂, 15 mmol/L Hepes. For I_{K1} measurements, 5 μmol/L nifedipine was added to block I_{CaL} and BaCl₂ 1 mmol/L were used to isolate I_{K1} from other background currents. I_{K1} was recorded using a step protocol with a holding potential of −50 mV and stepping from −120 to +20 mV in 10-mV increments of 500-ms duration at each potential as before (Milstein M L, et al., Proceedings of the National Academy of Sciences. 2012; 109 :E2134-E2143; and Dhamoon A S, et al., Circulation Research. 2004;94:1332-1339, herein incorporated by reference in their entireties).

### RT-PCR Analysis of Gene Expression

RNA was extracted from monolayers using Rneasy Mini Kit (Qiagen) according to manufacturer protocol. Samples were treated with DNase I (Invitrogen) for 15 min at room temperature to avoid genomic DNA contamination. Concentration and quality of RNA was monitored spectroscopically and reverse transcription was performed using a SuperScript III First-Strand Synthesis System (Invitrogen). cDNA was synthetized from 500 ng RNA by reverse transcription using reverse transcriptase, 200 U.A. (Invitrogen), in a 20 μL reaction volume including 0.05 μg/μL primers oligodT primers (Invitrogen), 0.5 mmol/L dNTPs (Promega) and 10 mmol/L buffer dNTP (Invitrogen). Quantitative RT-PCR was performed using TaqMan Universal PCR Master Mix (Applied Biosystems) and TaqMan probes for SCN5A, ITGA5, ITGA6, ITGB1 and PTK2. Two microliters of cDNA from reverse-transcription reaction were added as template for each Q-PCR. Expression of each transcript was normalized to the expression of glycerol phosphate dehydrogenase (GAPDH). Mean cycle threshold (Ct) value was first calculated and then ACt was calculated as each gene's mean Ct value minus the mean Ct value of the endogenous control. Fold of expression was calculated according to the formula: fold of expression=2(−ΔCt).

### Western Blotting

Immediately after optical mapping, cells were collected in Laemmli sample buffer and stored at −20° C. Standard western blotting techniques were used to quantify the relative levels of Cx43 (Rabbit polyclonal, Chemicon), Kir2.1 (mouse monoclonal, Neuromab) and kindlin-2 (antibody provided by Dr. James Dowling and has been validated using murine samples) (Dowling J J, et al., Circulation Research. 2008;102:423-431, herein incorporated by reference in its entirety). The total myosin (MF20, mouse monoclonal, ATCC) protein expression or GAPDH (rabbit polyclonal, Sigma) was used as the loading control in each case. Quantification was executed using ImageJ.

### Immunofluorescence

In a second set of experiments hiPSC-CM monolayers were fixed for immunocytochemistry following optical mapping recordings of impulse propagation as recently described (Lee P, et al., Circulation Research. 2012;110:1556-1563, herein incorporated by reference in its entirety). Briefly, cell monolayers were washed with PBS and then fixed with 4% paraformaldehyde for 10 minutes and rinsed twice before blocking with 10% normal donkey serum in PBS plus 0.1% Triton X-100 (Sigma) for 1.5 hours at room temperature. Primary antibodies were used to detect α-actinin (1:500, Sigma) in the cardiac sarcomeres and Cx43 (1:100, Millipore) and N-cadherin (1:200, BD Bioscience) at the intercellular junctions. The primary antibodies were added to 5% donkey serum in PBS plus 0.1% Triton X-100 and incubated overnight at 4° C. Subsequently, cells were washed three times in PBS plus 0.1% TRITON X-100. The secondary antibodies, donkey anti-rabbit DyLight 488 and donkey anti-mouse DyLight 549 (1:500, Jackson ImmunoResearch), diluted in the same solution as the primary antibodies, were applied to cells and incubated at room temperature in the dark for 1.5 hours. After incubation, cells were washed three times with PBS plus 0.1% TRITON X-100 and once with PBS only. Then, the nuclei were stained with DAPI (1:1000, Invitrogen) for 10 minutes in the dark at room temperature. Coverslips were mounted on slides for confocal imaging. Protein localization was examined by laser scanning confocal microscopy with sequential laser firing for multiple fluorophores (Nikon AIR, Melville, N.Y.). iPSC-CM cross sectional area was calculated in NIS Elements software using N-cadherin staining of the cell membranes.

### Cardiac Directed Differentiation of Other Human Pluripotent Stem Cell Lines

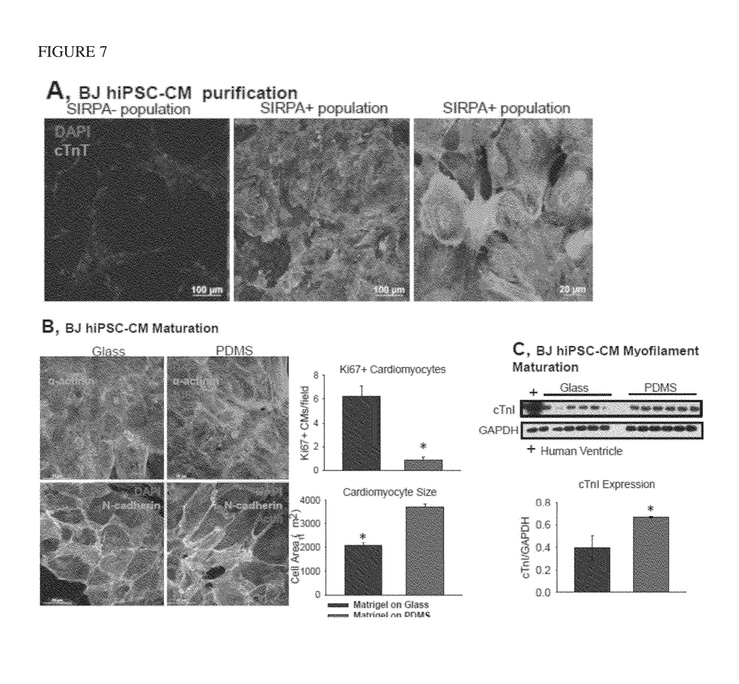

After optimization of the maturation process using iCell™ cardiomyocytes, the effect of soft PDMS substrate on the maturation state of cardiomyocytes derived from other human pluripotent stem cell lines was also determined. Human embryonic stem cell derived cardiomyocytes (ESC-CMs) were derived using the UM 22-2 (NIH registration #0209) generated in the laboratory of Dr. Gary Smith at the University of Michigan. Cardiac directed differentiation of UM 22-2 stem cells was accomplished in standard plastic 24-well dishes using the ‘matrix sandwich’ protocol of sequential cytokine and extracellular matrix application as described before (Zhang J, et al., Circulation Research. 2012;111:1125-1136, herein incorporated by reference in its entirety). ESC-CMs were purified using magnetic activated cell sorting (MACS) (Dubois N.C., et al., Nat Biotech. 2011;29:1011-1018, herein incorporated by reference in its entirety) on day 17 of directed differentiation and re-plated on matrigel coated glass or PDMS coverslips. On day 28, purified ESC-CMs were fixed and immunostaining for α-actinin and Cx43 was performed as described above (FIG. 7B). The recently described BJ-iPSC (Bizy A, et al., Stem Cell Research. 2013;11:1335-1347, herein incorporated by reference in its entirety) line generated by mRNA reprogramming was also used to test the effects of PDMS substrate on human iPSC-CM maturation. For this iPSC cell line cardiac directed differentiation was performed in standard plastic multi-well culture dishes using a small molecule based approach as described before (Lian X, et al., Proceedings of the National Academy of Sciences. 2012;109:E1848-E1857, herein incorporated by reference in its entirety). BJ-iPSC-CMs were MACS purified on day 20 and re-plated on matrigel coated glass or PDMS coverslips. BJ-iPSC-CM were then fixed in 3% paraformaldehyde on day 30 and immunostaining was performed as above for α-actinin, Ki67 (a cell proliferation marker), N-cadherin and sarcomeric actin as described above for the iCell™ cardiomyocytes (FIG. 7A).

Finally, the effect of soft PDMS substrate on the cardiac directed differentiation of the 19-9-11T hiPSC cell line developed by Yu et al. (Yu J, et al., Science. 2009;324:797-801, herein incorporated by reference in its entirety) was tested. Here iPSC monolayers were grown in 24 well culture dishes with or without custom fabricated PDMS coverslips lining the bottom of the well. Cardiac directed differentiation was performed using the highly efficient small molecule method (Lian X, et al., Proceedings of the National Academy of Sciences. 2012;109:E1848-E1857, herein incorporated by reference in its entirety). On day 30 19-9-11T cardiac monolayers were loaded with fluo-4AM for optical mapping of calcium impulse propagation as described above for the iCell™ cardiomyocytes. Conduction velocity was quantified for cardiac monolayers generated on PDMS and compared to those grown on standard plastic bottom multi-well dishes (FIG. 7C).

### Statistics

All data are presented as mean±SE. One way ANOVA with Bonferroni's multiple comparisons test was used for analysis of multiple groups. Student's t-test was used to determine statistical significance for data involving just two groups. P values are indicated for each Figure.

### EXAMPLE 1

### ECM Effects on hiPSC-CM Monolayer Impulse Propagation

FIG. 1 (left) demonstrates that purified hiPSC-CM cardiomyocytes cultured as monolayers on PDMS+matrigel achieve a high degree of electrical maturity, with average action potential propagation velocities as high as 55 cm s⁻¹. It is important to note however that while hiPSC-CM cardiomyocytes are highly purified; one always encounters mixtures of different cardiomyocyte phenotypes, including atrial-like, ventricular-like and pacemaker-like myocytes. This is reflected in FIG. 1A by the different optical action potential (AP) configurations (right) in the monolayer. Pacemaker-like cells at the site of impulse initiation undergo slow diastolic depolarization at a steady rate until the threshold potential is reached and an action potential is generated. More distally, ventricular-like APs have stable resting membrane potentials and respond to the propagating impulse with very rapid upstrokes. In FIGS. 1B and C, action potential propagation velocity and duration over electrical pacing frequencies ranging from 0.7 to 2.5 Hz were characterized. FIG. 1B shows conduction velocity restitution as one would expect with faster conduction at lower frequency (greater cycle length) of stimulation. FIG. 1C demonstrates the action potential duration restitution of mature hiPSC-CM monolayers where APD gets shorter as pacing frequency increases. Thus, the biomatrix combination of matrigel+PDMS promotes functional electrophysiological maturation of hiPSC-CMs in as little as four days.

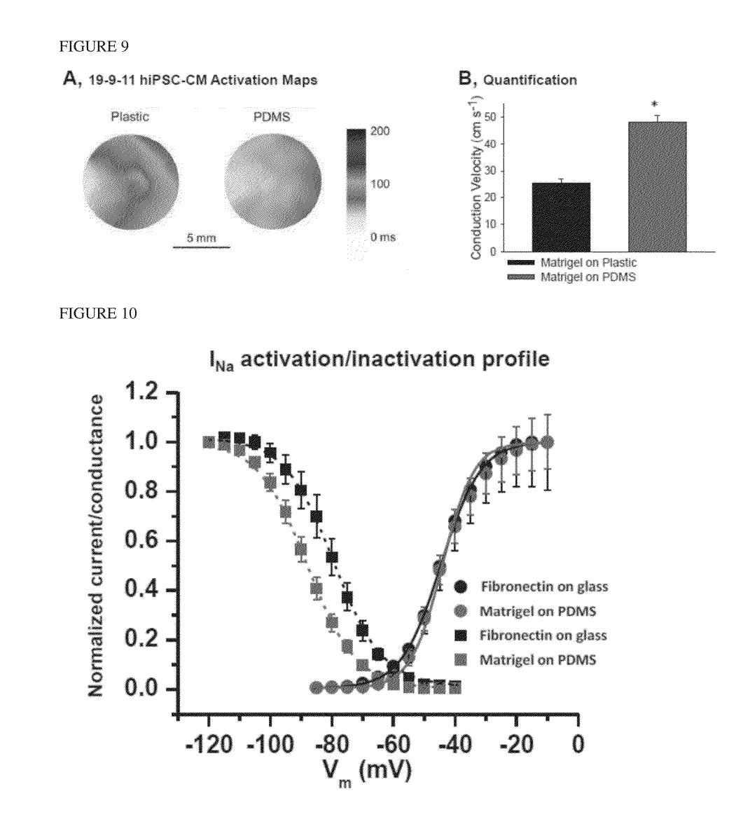

To establish whether the softer ECM provided by PDMS+matrigel promoted maturation significantly more than stiffer matrices, the combinations presented in FIG. 2A were tested by looking at calcium transient (CaT) propagation across the monolayer. First, the rate of spontaneous pacemaker activity was recorded for each of the 4 conditions. There was no difference in the spontaneous activation rate between the groups with the averages being the following: i. fibronectin+glass=0.25±0.05 Hz n=10, ii. fibronectin+PDMS=0.22±0.03 Hz n=4, iii. matrigel+glass=0.24±0.09 Hz n=14, and iv. matrigel+PDMS=0.2±0.07 Hz n=9 (mean±SEM, One way ANOVA, P>0.9999). Shown in panel B of FIG. 2 are representative color CaT activation maps of wave propagation for each of the experimental conditions. The fastest CV was observed in human cardiac monolayers cultured on matrigel+PDMS (FIG. 2B, iv). The quantification of CV in each condition is shown in panel C of FIG. 2. The average CVs were: fibronectin+glass=21.6±6.8 cm·s⁻¹ n=10, fibronectin+PDMS=24.6±6 cm·s⁻¹n=4, matrigel+glass=22.0±4.0 cm·s⁻¹ n=14, and matrigel+PDMS=43.6±7.0 cm·s⁻¹ n=9 (mean±SEM, One way ANOVA, see Figure and legend for details). Upper 95% confidence interval for the matrigel+PDMS group is 47.8 cm·s⁻¹. Point stimulation of monolayers (15-20V, 5 ms duration, 1 Hz) in each condition was performed in a separate group of experiments to determine differences in CV. The average CVs during 1 Hz pacing were: i. fibronectin+glass=24.2±1.8 cm·s⁻¹ n=6, ii. fibronectin+PDMS=28.1±1.5 cm·s⁻¹n=4, iii. matrigel+glass=28.4±3.2 cm·s⁻¹ n=5, and iv. matrigel+PDMS=37.1±1.7 cm·s⁻¹ n=6 (FIG. 2D, mean±SEM). Thus CV was faster during spontaneous pacemaker activations as well as during 1 Hz electrical pacing in human cardiac monolayers cultured on matrigel coated PDMS coverslips. Additionally, using 19-9-11T hiPSCs, the cardiac differentiation process (Lian X, et al., Proceedings of the National Academy of Sciences. 2012) was carried out on PDMS lined cell culture dishes and it was found that impulse propagation of hiPSC-CMs differentiated on PDMS was faster compared to hiPSC-CMs generated on plastic bottom dishes (FIG. 9).

### EXAMPLE 2

### ECM Effects on hiPSC-CM Monolayer Electrophysiology

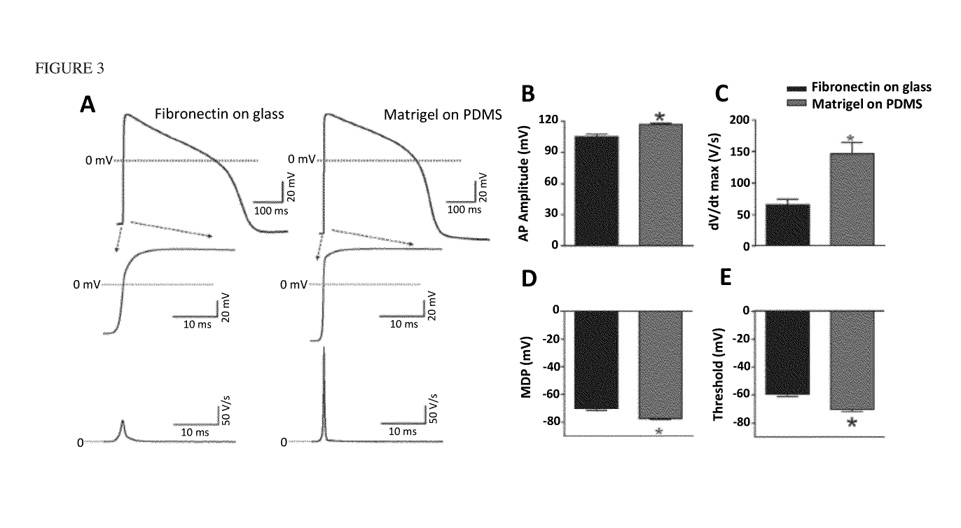

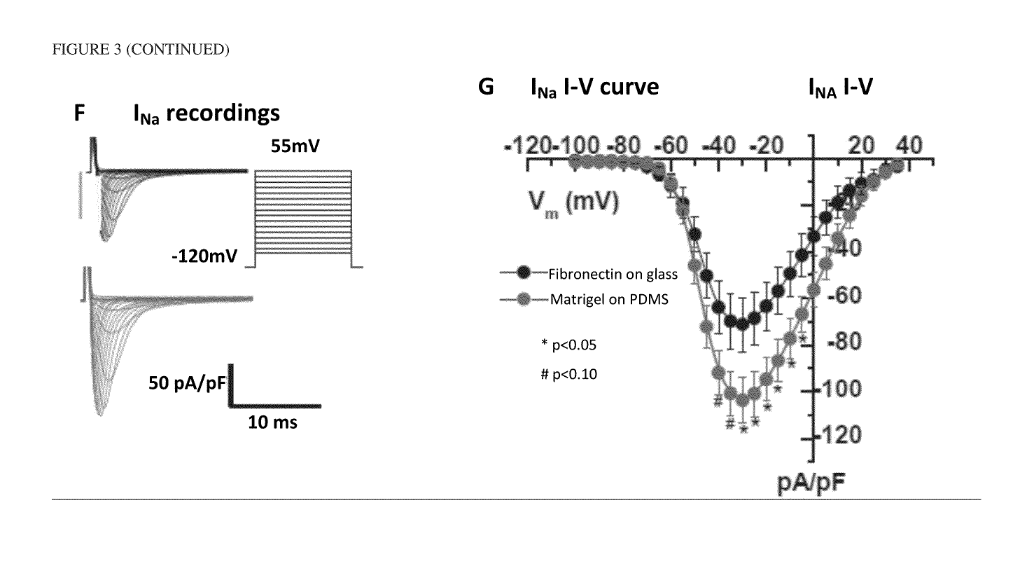

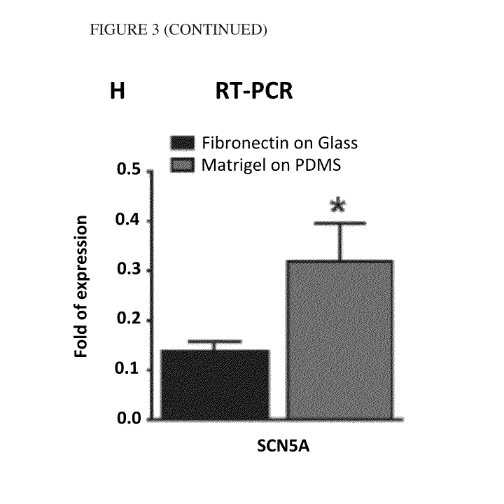

APs are required for propagation of the electrical signal that triggers the Ca²⁺ mediated excitation-contraction coupling (Ma et al., American Journal of Physiology—Heart and Circulatory Physiology. 2011;301:H2006-H2017; Mummery C L, et al., Circulation Research. 2012;111:344-358). Therefore, hiPSC-CM monolayers APs were recorded by patch-clamp analysis in current-clamp mode. Properties of the AP such as the dV/dtₘₐₓ (V/s, FIGS. 3A&C), the MDP (FIG. 3D), and the threshold potential (take-off potential, FIG. 3E) provide quantitative metrics of the degree of myocyte maturity (Ma et al., American Journal of Physiology—Heart and Circulatory Physiology. 2011;301:H2006-H2017); Yang X, et al., Circulation Research. 2014;114:511-523; Mummery C L, et al., supra). These parameters were measured in hiPSC-CMs cultured on the extremely rigid ECM condition of fibronectin+glass (FIG. 3) and compared the results to hiPSC-CMs cultured on the softest ECM condition of matrigel+PDMS (FIG. 3). APs recorded from hiPSC-CMs grown on matrigel+PDMS displayed significantly faster upstroke velocity (65.3±8.9 V/s, N=6 monolayers, n=37 vs. 146.5±17.7 V/s, N=5 monolayers, n=24; FIG. 3C), more hyperpolarized MDP (−69.9±1.7 mV, N=6 monolayers, n=37 vs. −77.5±0.6 mV, N=5 monolayers, n=24; FIG. 3D) and more hyperpolarized take-off potential (−59.3±1.7 mV, N=6 monolayers, n=37 vs. −70.5±1.2 mV, N=5 monolayers, n=24; FIG. 3E), all indicative of cardiomyocyte maturation (Robertson C, et al., STEM CELLS. 2013).

The faster dV/dtₘₐₓ and faster impulse propagation may be attributed partially to the effect of matrigel+PDMS ECM to increase sodium current density (I_{Na}). Indeed, single cell patch clamp analysis of I_{Na} revealed significantly elevated current density in cardiomyocytes cultured on PDMS+matrigel compared to the same batch of cardiomyocytes cultured on fibronectin on glass coverslips (FIG. 3F&G; glass N=4, n=12 and PDMS N=4, n=19). Elevated I_{Na} density observed here is consistent with previous data showing that I_{Na} density increases in the maturing heart (Davies M P, et al., Circulation Research. 1996;78:15-25). FIG. 10 shows the I_{Na} activation/inactivation profiles. The inactivation profile is left shifted for cardiomyocytes cultured on PDMS+matrigel (V1/2=−79.0±2.45 mV, N=4, n=13 vs. −88.0±1.55 mV, N=4, n=12; P=0.009), which indicates increased expression of cardiac sodium channel isoforms (i.e., SCN5A, Nav1.5) that is known to be larger in adult than immature embryonic cardiac myocytes (Davies et al., supra; Dominguez J N, et al., Cardiovasc Res. 2008;78:45-52). RT-PCR analysis confirmed elevated SCN5A gene (FIG. 3H) expression in iCell™ iPSC-CMs cultured on matrigel coated PDMS compared to iPSC-CMs cultured on fibronectin coated glass coverslips.

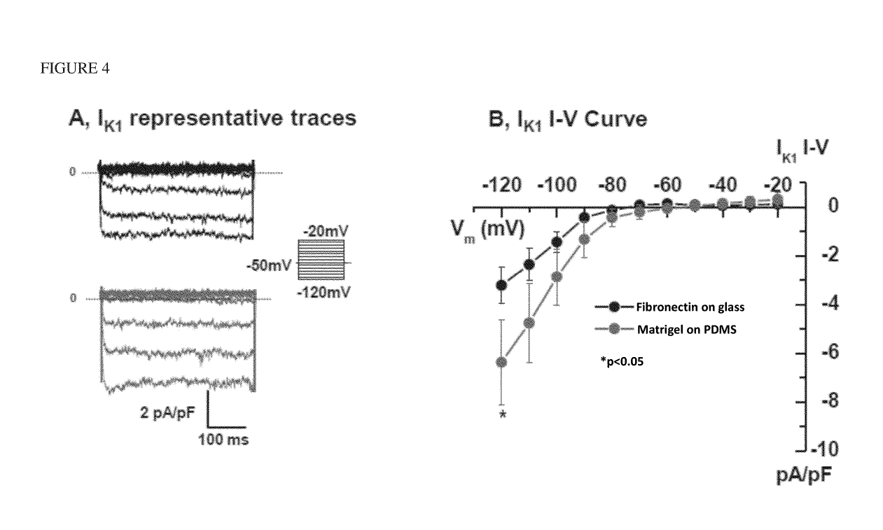

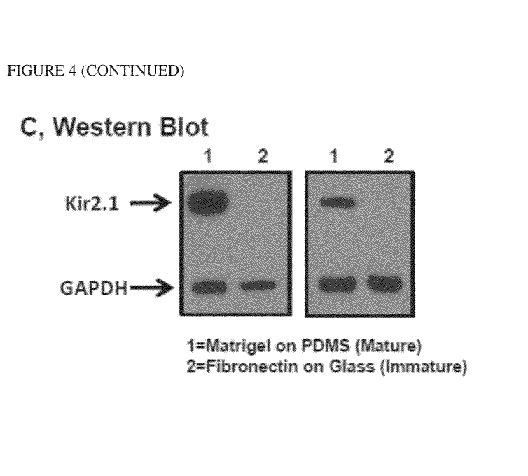

It has been reported that the MDP and spontaneous activity of hiPSC-CMs is critically dependent on the density of the rapid component of the delayed rectifier potassium current (I_{Kr}) and that I_{K1} density is extremely low if not absent in hiPSC-CMs (Doss M X. PLoS ONE. 2012;7:e40288). The hyperpolarized MDP of iCell™ cardiomyocytes cultured on PDMS+matrigel indicate that the I_{K1} density is relatively high. Indeed, patch clamp analysis revealed elevated I_{K1} density in cardiomyocytes maintained on PDMS+matrigel biomatrix (FIG. 4A&B). Western blotting shows expression of Kir2.1 exclusively in cardiomyocytes cultured on PDMS+matrigel compared to the same cells cultured on fibronectin coated glass coverslips (FIG. 4C).

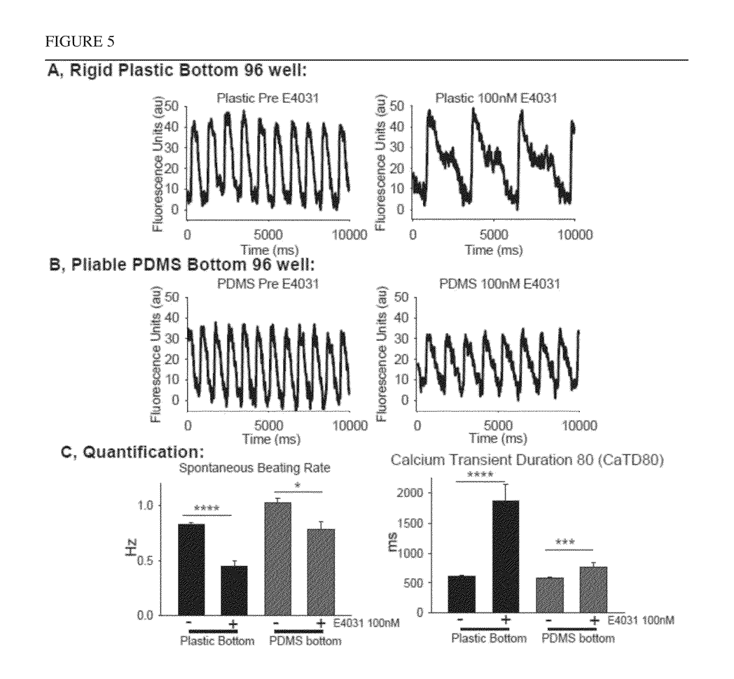

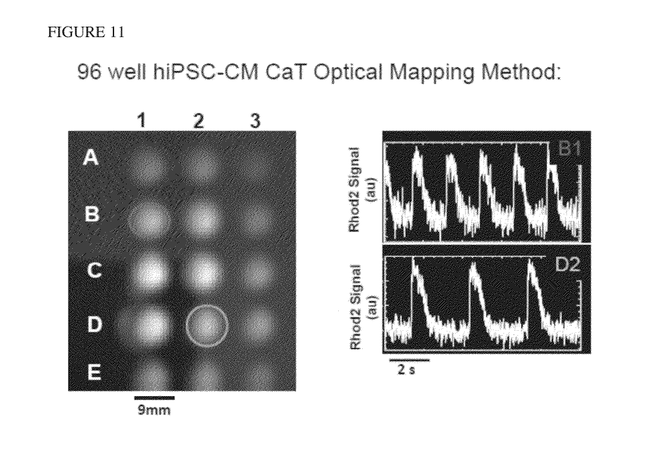

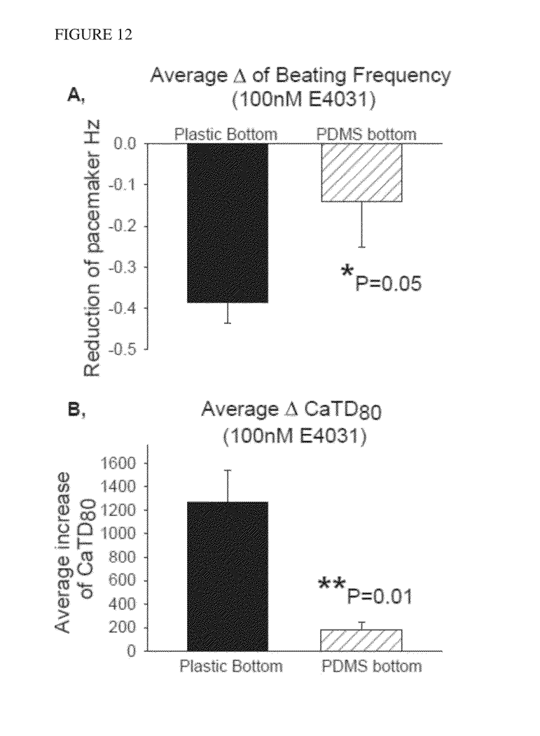

To further investigate whether PDMS+matrigel promotes the expression of other potassium currents, the effects of E4031 (100 nmol L⁻¹), a selective blocker of the rapid component of the delayed rectifier potassium current (I_{Kr}) on the spontaneous beating rate and calcium transient duration (CaTD₈₀) of hiPSC-CM monolayers cultured on matrigel coated PDMS was compared to those cultured on standard rigid, plastic bottom 96 well dishes (also matrigel coated, FIG. 11 illustrates how this multi-well optical mapping platform was used). Small glass coverslips to fit into a 96 well format dish are not readily available so here plastic was used as the rigid comparator for PDMS. FIGS. 5 and 12 each show that the electrophysiology of hiPSC-CM monolayers cultured on PDMS, measured by the CaT duration, is less affected by I_{Kr} blockade, further supporting the evidence in FIG. 4 of elevated potassium current densities in hiPSC-CMs cultured on matrigel coated PDMS coverslips. For example, 100 nmol L⁻¹ E4031 reduced spontaneous beating frequency by ˜50% (0.83±0.02 Hz pre E4031 vs. 0.45±0.05 Hz post E4031, n=8) in monolayers plated on rigid plastic, whereas the same dose of E4031 modestly reduced beating frequency by ˜20% (1.02±0.04 Hz pre E4031 vs. 0.78±0.07 Hz post E4031, n=5). This effect on beating frequency is due to the effect of of E4031 to prolong the action potential and calcium transient durations. Similarly, CaTD₈₀ increased ˜3× following E4031 application in hiPSC CMs plated on plastic bottom dishes (605.9±24.3 ms pre E4031 to 1873.8±278.9 ms post E4031, n=8), but CaTD₈₀ only increased by ˜1.34× in hiPSC CMs plated on PDMS bottom dishes (579.8±13.9 ms pre E4031 vs. 780.1±74.1 ms post E4031, n=5; FIG. 12).

### EXAMPLE 3

### ECM Effects on Intercellular Junction Formation Gap junctions: Cx43 Expression

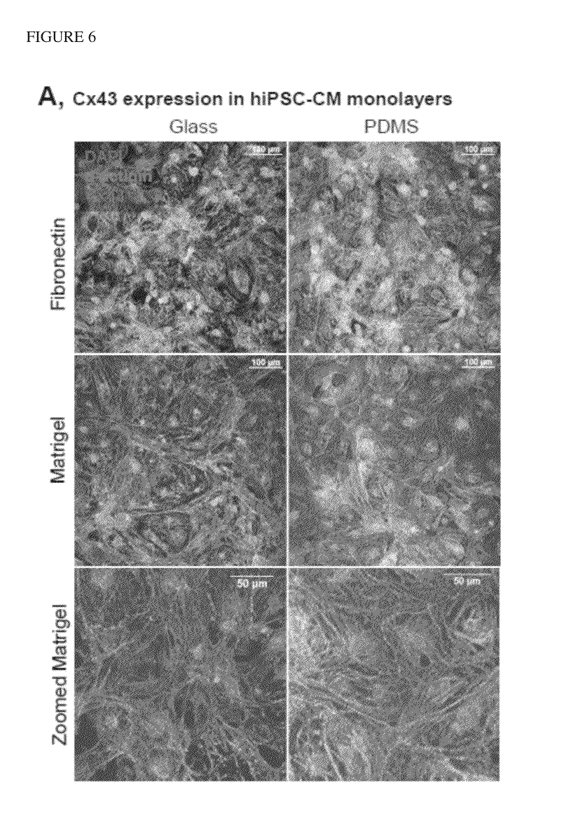

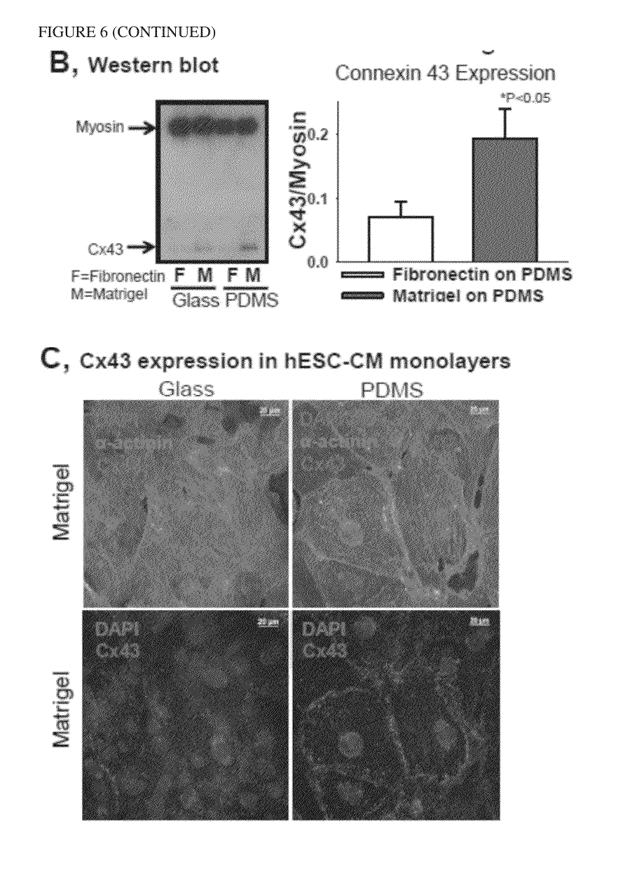

ECM stiffness has been shown to impact rodent neonatal myocyte Cx43 expression at the intercellular gap junctions. Previously, Forte et al. found that softer substrates impact Cx43 expression and myocyte morphology (Forte G, et al., Tissue Eng Part A. 2012;18:1837-1848). Consistent with previous reports using rodent myocytes, it was found that Cx43 expression is elevated in hiPSC-CM monolayers cultured on PDMS coated with matrigel compared to monolayers cultured on glass coverslips (FIG. 6) or fibronectin coated PDMS. The effect of the different plating combinations (FIG. 2A) on Cx43 expression and subcellular localization was determined. Panel A of FIG. 6 shows the Cx43 expression and localization in hiPSC-CM monolayers plated in the various ECM conditions. The greatest Cx43 expression at the intercellular junctions is found in monolayers plated on matrigel+PDMS. This provides another molecular mechanism to explain the faster CV found for this biomatrix combination. In FIG. 6B, Western blot analysis shows that the amount of Cx43 expression was ˜3× greater when fibronectin+PDMS is compared to matrigel+PDMS (0.07±0.05 A.U. vs. 0.19±0.10 A.U., n=5 monolayers, p<0.05). Next, the effect of PDMS to promote Cx43 expression in UM22-2 hESC-CM monolayers was determined. The UM22-2 control hESC line was derived in the laboratory of Dr. Gary Smith at the University of Michigan and cardiomyocytes were generated by the matrix sandwich differentiation protocol (Zhang J, et al., Circulation Research. 2012;111:1125-1136). Immunostaining for a-actinin and Cx43 in hESC-CM monolayers also indicated robust induction of Cx43 expression and localization at the cell-cell borders by PDMS substrate (FIG. 6C). Similar to the iCell™ Cx43 expression, hESC-CM Cx43 expression outlines the entire cardiomyocytes when cells are cultured on PDMS. Collectively, these results demonstrate that the ECM combination of matrigel+PDMS promotes the development of functional gap junctions available for more efficient intercellular communication and faster impulse propagation in hPSC-CM monolayers.

### EXAMPLE 4

### Adherens Junctions: N-Cadherin

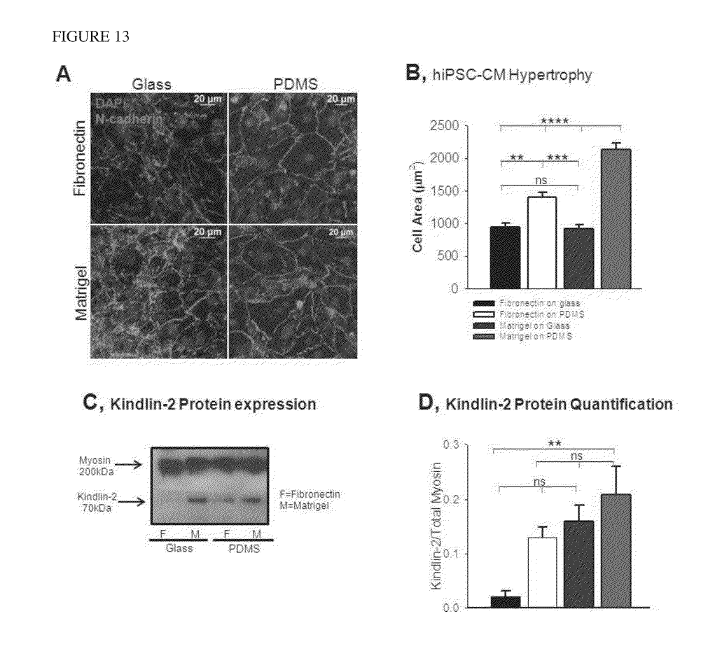

Adherens junction formation is a prerequisite for gap junction assembly in cultured adult rat cardiomyocytes (Lee P, et al., Heart Rhythm. 2011;8:1482-1491; and Hou L, et al., Circulation Research. 2010;107:1503-1511, herein incorporated by reference in their entireties). Adhesion among cardiac cells is mediated by transmembrane glycoproteins such as N-cadherin, a Ca²⁺-dependent adhesion molecule, which belongs to the cadherin superfamily (Leckband D, Prakasam A. Annual Review of Biomedical Engineering. 2006;8:259-287, herein incorporated by reference in its entirety). Thus, the results in FIG. 6 showing increased gap junction formation in hiPSC-CM monolayers cultured on matrigel+PDMS indicates that the mechanical adherens junctions may also be more fully developed in this case (Kostin S, et al., Circulation Research. 1999;85:154-167; and Zuppinger C, et al., Journal of Molecular and Cellular Cardiology. 2000;32:539-555, herein incorporated by reference in their entireties). Therefore, N-cadherin expression and localization in hiPSC-CM monolayers by immunocytochemistry and confocal imaging was determined (FIG. 13). The pattern of N-cadherin (green) staining at the intercellular junctions appears more organized at the cell-cell interface in monolayers plated on PDMS. This indicates more mature adherens junction formation and thus tighter electromechanical coupling between hiPSC-CM monolayers plated on soft substrates.

### EXAMPLE 5

### ECM promotes hiPSC-CM Hypertrophy and Elongation

After birth, myocytes in the heart switch from hyperplastic growth to hypertrophic growth (Li F, et al., J Mol Cell Cardiol. 1996;28:1737-1746, herein incorporated by reference in its entirety). Therefore another marker of maturation of hPSC-CMs is the transition from cardiomyocytes remaining in the cell cycle to myocyte terminal differentiation and hypertrophy. A commonly used cell cycle marker is Ki-67 expression in the nucleus of cardiomyocytes (Walsh S, et al., Cardiomyocyte cell cycle control and growth estimation in vivo—an analysis based on cardiomyocyte nuclei. 2010). Using highly purified BJ hiPSC-CMs (Bizy A, et al., Stem Cell Research. 2013;11:1335-1347) (FIG. 7A), the effect of PDMS to reduce the number of cardiomyocytes remaining in the cell cycle was quantitated (FIG. 7B). Additionally, the hiPSC-CMs cultured on pliable PDMS were significantly larger in size than cells plated on glass substrates (FIG. 7B and FIG. 13). This indicates a greater degree of terminal differentiation and physiological hypertrophy in hiPSC-CMs cultured on soft substrates. Binucleation, another marker of myocyte maturity (Li F, et al., Journal of Molecular and Cellular Cardiology. 1996;28:1737-1746; Katzberg A A, et al., American Journal of Anatomy. 1977;149:489-499), was also apparent in hiPSC-CMs cultured on matrigel+PDMS (FIG. 13). One key myofilament marker of cardiomyocyte maturation is cTnI expression (Yang X, et al., Circulation Research. 2014;114:511-523). Thus, Western Blot analysis of BJ hiPSC-CM expression of cTnI when purified monolayers were cultured on glass or PDMS coverslips coated with matrigel was performed. Significantly more robust cTnI expression was detected relative to GAPDH in purified BJ hiPSC-CMs plated on PDMS coverslips compared to glass coverslips (FIG. 7C), thus indicating a greater level of sarcomeric maturation and promotion of developmental isoform switching that is known to occur in the post-natal heart.

### EXAMPLE 6

### Maturation of Stem Cell Derived Cardiomyocytes From Other Pluripotent Stem Cell Lines

The effect of the softer PDMS substrate on the maturation of cardiomyocytes derived from two other human iPSC lines and one human ESC line was tested. FIG. 7B shows the effect of PDMS on the maturation state of purified BJ-iPSC-CMs. Ki67 immuno-reactivity is a marker of cell proliferative activity. Fewer BJ-iPSC-CMs were positive for a-actinin and Ki67 when grown on PDMS coverslips compared to glass coverslips (FIG. 7B top panels). This indicates more terminal differentiation and maturation of iPSC-CMs grown on PDMS coverslips. Furthermore, immunostaining for N-cadherin and sarcomeric actin shows that PDMS also promoted the hypertrophy and elongation of BJ-iPSC-CMs (FIG. 7B, bottom panels). This is consistent with the hypertrophy and elongation observed in the iCell™ cardiomyocytes cultured on PDMS. ESC-CMs were also purified and replated on glass or PDMS coverslips using the same protocol as for the BJ-iPSC-CMs. Immunostaining for a-actinin and Cx43 in ESC-CM monolayers indicates induction of Cx43 expression by PDMS substrate (FIG. 6C). Similar to the iCell™ Cx43 expression, ESC-CM Cx43 expression outlines the entire cardiomyocytes when cells are cultured on PDMS.

Finally cardiac directed differentiation of the 19-9-11T iPSC line was executed on PDMS coverslips. Results were compared to 19-9-11T iPSC-CMs differentiated on conventional plastic bottom culture dishes. Optical mapping of calcium impulse conduction velocity served as an indication of the level of cardiac monolayer maturity. Monolayers generated on PDMS had significantly faster pacemaker impulse conduction velocity compared to monolayers generated on plastic. FIG. 9 shows example activation maps and quantification of the conduction velocity in each condition.

### Integrin Signaling in the Maturation Process

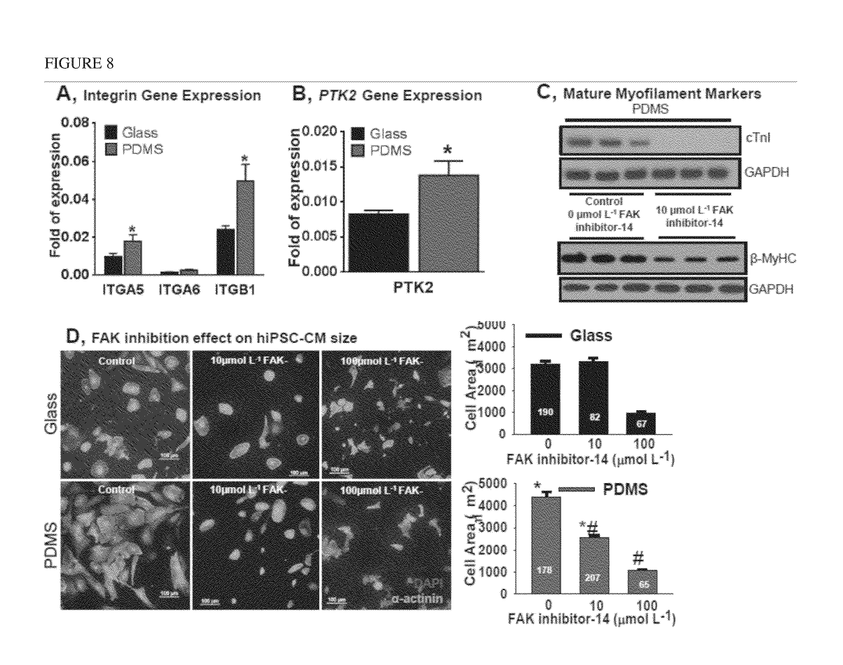

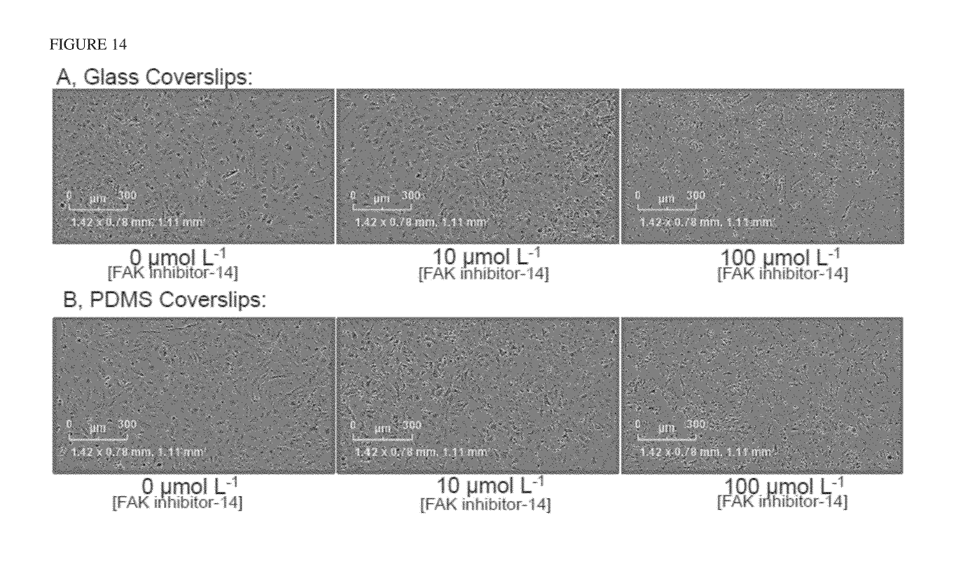

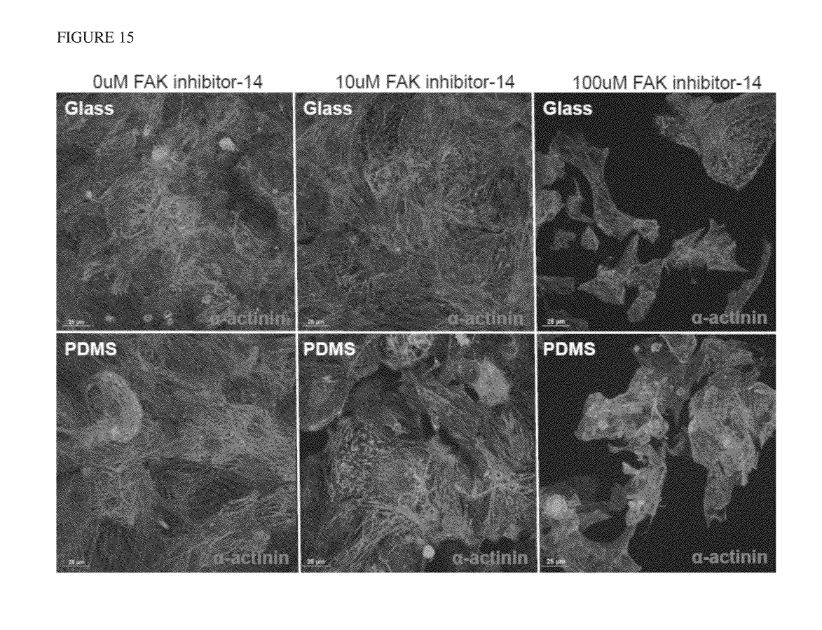

Integrins are transmembrane heterodimeric receptors essential for providing cell-extracellular matrix adhesion, cellular structural organization and transduction of mechanical signals from the extracellular matrix into biochemical signals in cardiomyocytes (Ross R S, et al., Circulation Research. 2001;88:1112-1119; Israeli-Rosenberg S, et al., The FASEB Journal. 2015;29:374-384; Israeli-Rosenberg S, et al., Circulation Research. 2014;114:572-586). β1 integrins are abundant in the adult heart and participate in the hypertophic response in rodent ventricular myoyctes (Ross et al., Circulation Research. 1998;82:1160-1172). Therefore, it was contemplated that integrin signaling underlies the maturation of hPSC-CM monolayers induced by the cell culture condition of plating PSC-CMs on matrigel coated PDMS coverslips. First RT-PCR analysis showed that ITGB1 expression is significantly induced on PDMS coverslips compared to glass coverslips (FIG. 8A). Additionally, PTK2 gene expression is elevated in hiPSC-CM monolayers cultured on PDMS coverslips (FIG. 8B). The PTK2 gene encodes the Focal Adhesion Kinase (FAK) intracellular molecule that is a primary mediator of integrin signaling (Cheng et al., Journal of Molecular and Cellular Cardiology. 2014;67:1-11). Purified hiPSC-CMs (iCell™) cultured on PDMS in the presence of 10 μmol L⁻¹ FAK inhibitor-14 failed to express cTnI and also expressed less β-MyHC (FIG. 8C). This indicates that FAK activation underlies expression of mature myofilament markers. Finally, the role of FAK activation in the hypertrophic response of hiPSC-CMs to the soft cell culture environment on PDMS was determined (FIG. 8D and FIG. 14). FAK inhibitor-14 prevented hypertrophic growth of hiPSC-CMs observed on PDMS coverslips. hiPSC-CMs grown on glass coverslips were only affected at very high dose of FAK inhibitor-14(100 μmol L⁻¹). In both conditions (Glass and PDMS) 100 μmol L⁻¹ FAK inhibitor-14 prevented monolayer formation and hiPSC-CM expansion in culture (FIG. 8D and FIGS. 14 and 15).

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in the art are intended to be within the scope of the following claims.

We claim:

1. A method for preparing cardiomyocytes, comprising: culturing cells on a flexible surface coated with extracellular matrix proteins under conditions such that cardiomyocytes are generated.

2. The method of claim 1, wherein said cells are stem cells.

3. The method of claim 1, wherein said cells are induced pluripotent cell cardiomyocytes.

4. The method of claim 1, wherein said culturing comprises culturing an interconnected monolayer of said cells.

5. The method of claim 2, wherein the stem cells are induced pluripotent stem cells (iPSCs).

6. The method of claim 5, wherein the iPSCs are human iPSCs.

7. The method of claim 2, wherein the stem cells are embryonic stem cells.

8. The method of claim 1, wherein said flexible surface is a coverslip.

9. The method of claim 1, wherein said flexible surface is a 96 well plate.

10. The method of claim 1, wherein said flexible surface comprises a silicone film.

11. The method of claim 10, wherein said silicone film comprises a polydimethylsiloxane (PDMS) film.

12. The method of claim 1, wherein said extracellular matrix proteins are provided by a matrigel coating.

13. The method of claim 1, wherein the cardiomyocytes have an action potential upstroke velocity greater than 100 V/s.

14. The method of claim 1, wherein the cardiomyocytes have a hyperpolarized diastolic membrane potential.

15. The method of claim 1, wherein the cardiomyoctyes have a propagation velocity greater than 25 cm s⁻¹.

16. The method of claim 1, wherein the cardiomyocytes are generated in a period less than 2 weeks.

17. A composition comprising a plurality of cardiomyocytes produced by the method of claim 1.

18. A method for testing a compound, comprising: a) providing a composition of claim 17; b) exposing a test compound to said composition; and c) determining an effect of said test compound on said composition.

19. The method of claim 18, wherein said test compound comprises a candidate therapeutic compound.

20. The method of claim 18, wherein said effect is one or more cardiac electrophysiological functions selected from the group consisting of action potential duration, beating frequency, conduction velocity and intracellular calcium flux amplitudes.
